(19) United States
(12) Patent Application Publication
(10) Pub. No.: US 2014/0045999 A1
(43) Pub. Date: Feb. 13, 2014

(54) MEDICAL RUBBER

(71) Applicant: SUMITOMO RUBBER INDUSTRIES, LTD., Kobe-shi (JP)

(72) Inventors: Kazuo FUTAMATA, Kobe-shi (JP); Kazuo Hochi, Kobe-shi (JP); Tatsuya Kubo, Kobe-shi (JP)

(73) Assignee: SUMITOMO RUBBER INDUSTRIES, LTD., Kobe-shi (JP)

(21) Appl. No.: 13/963,004

(22) Filed: Aug. 9, 2013

(30) Foreign Application Priority Data
Aug. 10, 2012 (JP) 2012-178391
Oct. 10, 2012 (JP) 2012-225296

(51) Int. Cl. A61L 31/04

(52) U.S. Cl. CPC A61L 31/049
USPC 525/256; 525/331.7

(57) ABSTRACT

The present invention provides a medical rubber having high cleanliness and excellent compression set resistance. The present invention relates to a medical rubber including an ethylene-propylene-diene rubber crosslinked by an organic peroxide having no aromatic ring structure.

### TECHNICAL FIELD

The present invention relates to a medical rubber.

### BACKGROUND ART

High cleanliness is demanded of medical rubber products. Specifically, the medical rubber products need to meet the requirements specified in the section of Extractable substances in the Test for Rubber Closure for Aqueous Infusions in the Japanese Pharmacopoeia, for example, the medical rubber product is required not to contain more than prescribed amounts of substances to be detected when it is leached in pure water.

Known examples of such medical rubber products include conventional crosslinked rubbers obtained by a crosslinking step using a crosslinking agent such as sulfur or a thiuram compound and the like to give rubber elasticity. Unfortunately, due to residues of a crosslinking agent and a crosslinking accelerator and decomposition products of the polymer, these crosslinked rubbers contain large amounts of organic substances to be detected in the tests for extractable substances. Moreover, halogenated butyl rubbers are also proposed, but may have an environmental impact because they contain halogens.

Meanwhile, thermoplastic elastomers (TPE) that do not need the crosslinking process, thermoplastic elastomers that involve dynamic vulcanization (TPV), and the like have also been developed. These elastomers do not need the crosslinking process and thus can avoid as poor results in the tests for extractable substances as the results of the crosslinked rubbers. These elastomers, however, are disadvantageously inferior in heat resistance and compression set resistance because they have no chemical crosslinking point and are thermoplastic. Consequently, it is desired to provide medical rubber products having high cleanliness, good heat resistance, and good compression set resistance, and further having no environmental impact.

### SUMMARY OF INVENTION

### Technical Problem

An object of the present invention is to solve the problems above and provide a medical rubber having high cleanliness and excellent compression set resistance.

### Solution to Problem

The present invention relates to a medical rubber, comprising an ethylene-propylene-diene rubber crosslinked by an organic peroxide (A) having no aromatic ring structure.

The medical rubber is preferably subjected to secondary crosslinking.

The medical rubber is preferably obtained by crosslinking an ethylene-propylene-diene rubber in the presence of a polyfunctional monomer (B) and zinc white (C) by the organic peroxide (A) having no aromatic ring structure, and further performing secondary crosslinking.

A diene component in the ethylene-propylene-diene rubber is preferably derived from ethylidene norbornene.

An ethylidene norbornene content is 6 to 14% by mass.

The organic peroxide (A) is preferably at least one selected from the group consisting of compounds respectively represented by the following formulas (1), (2), and (3):

(H₃C₃)₃C—O—O—R¹¹—O—O—C(CH₃)₃ (1)

wherein R¹¹ represents a saturated divalent hydrocarbon group optionally containing a substituent;

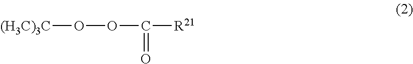

wherein R²¹ represents a saturated monovalent hydrocarbon group or a saturated alkoxy group; and

(H₃C₃)₃C—O—O—C(CH₃)₃ (3).

The substituent is preferably a group represented by —C(═O)—O—R¹² wherein R¹² is a saturated monovalent hydrocarbon group.

Preferably, 0.3 to 15 parts by mass of the organic peroxide (A) is contained per 100 parts by mass of the ethylene-propylene-diene rubber.

The polyfunctional monomer (B) is preferably at least one selected from the group consisting of di- or triallyl compounds, di(meth)acrylates, tri(meth)acrylates, divinyl compounds, and maleimide compounds.

Preferably, 0.5 to 10 parts by mass of the polyfunctional monomer (B) is contained per 100 parts by mass of the ethylene-propylene-diene rubber.

Preferably, 0.5 to 10 parts by mass of the zinc white (C) is contained per 100 parts by mass of the ethylene-propylene-diene rubber.

The medical rubber is preferably obtained by performing the secondary crosslinking for 1 hour or more. The medical rubber is preferably in conformity with the standards for extractable substances specified in the Japanese Pharmacopoeia, Sixteenth Edition.

### Advantageous Effects of Invention

The present invention provides a medical rubber including an ethylene-propylene-diene rubber crosslinked by an organic peroxide (A) having no aromatic ring structure. The medical rubber attains high cleanliness and excellent compression set resistance.

### DESCRIPTION OF EMBODIMENTS

The medical rubber according to the present invention includes an ethylene-propylene-diene rubber (EPDM) crosslinked by an organic peroxide (A) having no aromatic ring structure.

By crosslinking EPDM with an organic peroxide (A) not having any aromatic ring represented by the formulas (1), (2) and the like, it is possible to provide high cleanliness in conformity with the standards for extractable substances specified in the Pharmacopoeia, and at the same time provide excellent compression set resistance. Moreover, since the medical rubber is obtained by crosslinking EPDM by a specific organic peroxide, such a rubber has excellent heat resistance. When the medical rubber contains no halogen atom, such a rubber can also be provided as an environmentally desirable product.

Particularly, the medical rubber according to the present invention is preferably obtained by crosslinking an ethylene-propylene-diene rubber (EPDM) in the presence of a polyfunctional monomer (B) and zinc white (C) by the organic peroxide (A) having no aromatic ring structure, and further performing secondary crosslinking.

A medical rubber having high cleanliness in conformity with the standards for extractable substances in the Pharmacopoeia can be produced by crosslinking EPDM by the organic peroxide not having any aromatic ring represented by the formulas (1), (2), and the like; however, it is difficult to provide sufficiently satisfactory compression set resistance to the medical rubber. In the present invention, when EPDM, in the presence of a polyfunctional monomer and zinc white, is crosslinked by the organic peroxide and is further subjected to secondary crosslinking, it is possible to attain not only high cleanliness but also excellent compression set resistance. Moreover, since the medical rubber is obtained by crosslinking EPDM in the presence of a polyfunctional monomer and zinc white by a specific organic peroxide, such a rubber has excellent heat resistance. When the medical rubber contains no halogen atom, such a rubber can also be provided as an environmentally desirable product.

In the present invention, EPDM is used as the rubber component. This provides excellent gas barrier properties, heat resistance, and chemical resistance. Known EPDMs can be used. Examples of these EPDMs include ethylene-propylene-diene terpolymers obtained by copolymerizing a copolymer of ethylene and propylene with a diene component to introduce an unsaturated bond. These EPDMs may be used singly or in combinations of two or more.

The diene component used for EPDM is not particularly limited. The diene component typically has approximately 5 to 20 carbon atoms. Specific examples of the diene component include cyclic dienes such as 5-ethylidene-2-norbornene (ethylidene norbornene), 5-propylidene-5-norbornene, dicyclopentadiene, 5-vinyl-2-norbornene, 5-methylene-2-norbornene, 5-isopropylidene-2-norbornene, and norbornadiene; and acyclic non-conjugated dienes such as 1,4-pentadiene, 1,4-hexadiene, 4-methyl-1,4-hexadiene, 5-methyl-1,4-hexadiene, 1,5-hexadiene, 2,5-dimethyl-1,5-hexadiene, 5-methyl-1,5-heptadiene, 6-methyl-1,5-heptadiene, and 6-methyl-1,7-octadiene. Among these, cyclic dienes are preferred, and 5-ethylidene-2-norbornene is particularly preferred, from the viewpoint of cleanliness and compression set resistance. These may be used singly or in combinations of two or more.

The diene component content, based on 100% by mass of the total raw materials that form the EPDM, is preferably 6 to 14% by mass, and more preferably 8 to 13% by mass. A content of less than 6% by mass leads to a smaller degree of crosslinking, which may result in reduced hardness and dimensional stability. A content of more than 14% by mass may cause deterioration in heat resistance, chemical resistance, fatigue resistance, and the like. The EPDM may be a mixture of EPDMs having different diene contents. In this case, the diene component content refers to the average diene component content of all EPDMs. An EPDM other than those having a diene content of 6 to 14% by mass may be mixed as long as the average content falls within the range above.

The ethylene content, based on 100% by mass of the total raw materials that form the EPDM, is preferably 35 to 70% by mass, and more preferably 40 to 60% by mass. A content of less than the lower limit thereof may lead to a reduction in the mechanical strength of the rubber composition. A content of more than the upper limit thereof may lead to poor elongation.

The EPDM preferably has a Mooney viscosity (ML_{1′4} at 125° C.) of 5 to 100, more preferably 7 to 90, and still more preferably 10 to 85. A Mooney viscosity of less than the lower limit may lead to difficulties to disperse filler in the rubber, which may reduce mechanical strength. A Mooney viscosity of more than the upper limit may reduce kneading properties and molding properties.

The Mooney viscosity refers to the viscosity of a raw rubber measured with a Mooney viscometer.

In the present invention, EPDM is contained as the rubber component; moreover, other rubber materials may be contained in the range in which the effects of the present invention are not inhibited. Examples of other rubber materials include natural rubber, styrene-butadiene copolymer rubber, chloroprene rubber, hydrogenated nitrile-butadiene rubber, alkylated chlorosulfonated polyethylenes, isoprene rubber, epichlorohydrin rubber, butyl rubber, and acrylic rubber. For the effects of the present invention, the content of EPDM, based on 100% by mass of the rubber component, is preferably 90% by mass or more, more preferably 95% by mass or more, and particularly preferably 100% by mass.

The present invention uses an organic peroxide (A) having no aromatic ring structure for crosslinking of EPDM. This can prevent decomposition residues having an aromatic ring structure from eluting to give a UV absorption amount exceeding a prescribed value in the Pharmacopoeia test, and therefore allows for high cleanliness. In addition, excellent compression set resistance is also attained.

The organic peroxide (A) having no aromatic ring structure may suitably be at least one selected from the group consisting of compounds respectively represented by the following formulas (1), (2), and (3). This significantly improves cleanliness and compression set resistance so that the effects of the present invention can be sufficiently attained.

(H₃C₃)₃C—O—O—R¹¹—O—O—C(CH₃)₃ (1)

(wherein R¹¹ represents a saturated divalent hydrocarbon group optionally containing a substituent);

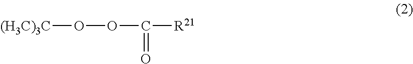

(wherein R²¹ represents a saturated monovalent hydrocarbon group or a saturated alkoxy group); and

(H₃C₃)₃C—O—O—C(CH₃)₃ (3)

(di-tert-butyl peroxide).

In the formula (1), the saturated divalent hydrocarbon group optionally containing a substituent as R¹¹ is preferably a C1-C10 alkylene group optionally containing a substituent, and may be any of linear, branched, and cyclic groups. Specific examples thereof include linear or branched alkylene groups such as a methylene group, an ethylene group, a propylene group, an n-butylene group, an i-butylene group, a pentylene group, a hexylene group, a heptylene group, and an octylene group; cycloalkylene groups (cyclic alkylene groups) such as a cyclohexylene group; and these groups containing substituents.

The substituent in R¹¹ is not particularly limited, and is preferably a group represented by —C(═O)—O—R¹² wherein R¹² represents a saturated monovalent hydrocarbon group. The saturated monovalent hydrocarbon group R¹² is preferably a C1-C10 alkyl group, and may be any of linear, branched, and cyclic groups. Specific examples thereof include a methyl group, an ethyl group, an n-propyl group, an isopropyl group, an n-butyl group, a t-butyl group, a pentyl group, a hexyl group, a heptyl group, an octyl group, and a nonyl group.

In the formula (2), the saturated monovalent hydrocarbon group as R²¹ is preferably a C1-C10 alkyl group, and may be any of linear, branched, and cyclic groups. Specific examples thereof include groups as mentioned for R¹². Examples of the saturated monovalent alkoxy group as R²¹ include alkoxy groups corresponding to the saturated monovalent hydrocarbon groups, and specifically include a methoxy group, an ethoxy group, an n-propoxy group, an n-butoxy group, an isobutoxy group, a sec-butoxy group, a tert-butoxy group, a hexoxy group, and an octoxy group.

Examples of the organic peroxides represented by the formula (1) include 1,1-di(t-butylperoxy)-2-methylcyclohexane, 1,1-di(tert-butylperoxy)cyclohexane, 1,1-di-(tert-butylperoxy)-3,3,5-trimethylcyclohexane, 2,2-di(tert-butylperoxy)butane, n-butyl-4,4-di(tert-butylperoxy)valerate, and 2,5-dimethyl-2,5-di(tert-butylperoxy)hexane.

Examples of the organic peroxides represented by the formula (2) include tert-butyl peroxyneodecanoate, t-butyl peroxyneoheptanoate, tert-butyl peroxy-2-ethylhexanoate, t-butyl peroxy-3,5,5-trimethylhexanoate, t-butyl peroxylaurate, tert-butyl peroxy isopropyl monocarbonate, t-butyl peroxy 2-ethylhexyl monocarbonate, and tert-butyl peroxyacetate.

The organic peroxide having no aromatic ring structure is more preferably an organic peroxide not containing any unsaturated bonds (C═C, C═O, and C═C). Organic peroxides containing an unsaturated bond can easily form compounds such as alcohol (OH) and aldehyde (CHO) as decomposition residues, and may lead to test results exceeding a prescribed value in the test for potassium permanganate-reducing substances.

The organic peroxide (A) having no aromatic ring structure is more preferably a compound represented by the formula (1) wherein R¹¹ is a saturated divalent hydrocarbon group. Particularly, 2,5-dimethyl-2,5-di(tert-butylperoxy)hexane, 2,2-di(tert-butylperoxy)butane, di-tert-butyl peroxide, and the like are preferred for a good balance between the crosslinking rate and the degree of crosslinking. These organic peroxides having no aromatic ring structure may be used singly or in combinations of two or more.

The amount of the organic peroxide (A) having no aromatic ring structure to be added is preferably 0.3 to 15 parts by mass, more preferably 0.3 to 10 parts by mass, further preferably 1 to 8 parts by mass, and still more preferably 2 to 6 parts by mass, per 100 parts by mass of the rubber component. With an amount of less than 0.3 parts by mass, sufficient hardness is unlikely to be obtained and dimensional accuracy and sealing properties tend to reduce. With an amount of more than 15 parts by mass, the rubber is likely to become excessively hard, and therefore sealing properties, flex resistance, and abrasion resistance as well as cleanliness tend to reduce.

In the case where the medical rubber according to the present invention is obtained by crosslinking an ethylene-propylene-diene rubber (EPDM) in the presence of a polyfunctional monomer (B) and zinc white (C) by an organic peroxide (A) having no aromatic ring structure, and further performing secondary crosslinking, the polyfunctional monomer (B) is a monomer having two or more non-conjugated double bonds per molecule. Examples of the monomer include di- or triallyl compounds, di(meth)acrylates, tri(meth)acrylates, divinyl compounds, and maleimide compounds. The addition of the polyfunctional monomer (B) can further reduce the compression set.

Examples of the di- or triallyl compounds include diallyl phthalate, diallyl maleate, diallyl fumarate, diallyl succinate, triallyl isocyanurate, triallyl cyanurate, and triallyl trimellitate. Examples of the di(meth)acrylates include ethylene glycol di(meth)acrylate, diethylene glycol di(meth)acrylate, triethylene glycol di(meth)acrylate, 1,6-hexane diol di(meth)acrylate, and trimethylolpropane di(meth)acrylate. Examples of the tri(meth)acrylates include trimethylolpropane tri(meth)acrylate, ethylene oxide modified trimethylolpropane tri(meth)acrylate, and pentaerythritol tri(meth)acrylate. Examples of the divinyl compounds include divinylbenzene and butadiene. Examples of the maleimide compounds include N-phenyl maleimide and N,N′-m-phenylene bismaleimide. Among these, the di- or triallyl compounds are preferred, triallyl compounds are more preferred, and triallyl isocyanurate is particularly preferred. These polyfunctional monomers can be used singly or in combinations of two or more.

The content of polyfunctional monomer is preferably 0.5 to 10 parts by mass, more preferably 1 to 8 parts by mass, and still more preferably 2 to 6 parts by mass, per 100 parts by mass of EPDM. With a content of less than 0.5 parts by mass, sufficient compression set resistance is unlikely to be obtained and dimensional stability and product durability tend to reduce. With a content of more than 10 parts by mass, cleanliness tends to reduce.

In the case where the medical rubber according to the present invention is obtained by crosslinking an ethylene-propylene-diene rubber (EPDM) in the presence of a polyfunctional monomer (B) and zinc white (C) by an organic peroxide (A) having no aromatic ring structure, and further performing secondary crosslinking, zinc white can be added to suppress degradation of the crosslinked rubber during secondary crosslinking. Examples of the zinc white include commercially available zinc white particles and the like. For example, zinc white particles having a particle size of 0.01 to 1.0 μm can be used, and those having a particle size of 0.05 to 0.25 μm can also be suitably used. Active zinc white having a smaller particle size of around 0.1 μm and having a significantly high activity, compared with typical zinc white having a particle size of 0.3 to 0.7 μm, can also be used in the present invention.

The particle size of zinc white can be measured by observing the particles with an electron microscope.

The content of zinc white is preferably 0.5 to 10 parts by mass, more preferably 1 to 8 parts by mass, and still more preferably 2 to 6 parts by mass, per 100 parts by mass of EPDM. With a content of less than 0.5 parts by mass, the effect of suppressing degradation of the crosslinked rubber tends not to be sufficiently obtained. With a content of more than 10 parts by mass, cleanliness tends to reduce.

Besides the components above, the medical rubber according to the present invention may incorporate a filler, a plasticizer, a processing aid, an antioxidant, an ultraviolet absorbing agent, and others commonly used for rubber, but these additives are desirably used in minimum amounts in order to balance cleanliness and physical properties because they have a great influence on cleanliness.

For dynamically used parts that repeatedly deform and contact, e.g., diaphragms, use of a filler is preferred because abrasion resistance is then improved. Examples of the filler include inorganic fillers such as calcium carbonate, silica, barium sulfate and talc, and carbon black.

The amount of the filler to be added per 100 parts by mass of the rubber component is preferably 70 parts by mass or less, more preferably 60 parts by mass or less, and is preferably 20 parts by mass or more, and more preferably 30 parts by mass or more, for a balance between abrasion resistance and cleanliness. With an amount of more than 70 parts by mass, cleanliness tends to reduce, and flex fatigue resistance also tends to reduce. With an amount of less than 20 parts by mass, abrasion resistance tends to become insufficient, thereby shortening product life.

Examples of the plasticizer include mineral oils and low molecular weight polymers such as liquid polyisobutylene. Use of a plasticizer having an aromatic ring structure, such as aromatic oil, is not preferred because it reduces cleanliness.

The components mentioned above are kneaded using an internal mixer such as an intermix, a Banbury mixer, and a kneader or an open roll mill, for example, whereby the medical rubber according to the present invention can be prepared. Moreover, the medical rubber of the present invention can be crosslinking molded at a temperature of 150 to 220° C. for approximately 0.5 to 60 minutes by, for example, compression molding or transfer molding, which include a press process or the like, or injection molding.

The medical rubber according to the present invention is preferably produced by performing not only the crosslinking molding but also secondary crosslinking in an oven or the like for improvement in cleanliness (the level of conformity with the Pharmacopoeia). The secondary crosslinking means a heat treatment of the crosslinked rubber in an oven or the like, and can decrease low molecular weight compounds such as residues and decomposition products of the polymer in the crosslinked rubber to enhance cleanliness.

The secondary crosslinking is preferably performed at a high temperature for a long period of time, but degradation of the crosslinked rubber may then be promoted. For this reason, the secondary crosslinking temperature is preferably 160° C. or less, more preferably 150° C. or less, and still more preferably 140° C. or less. The secondary crosslinking time is preferably as short as possible from the viewpoint of degradation of the crosslinked rubber and economy although it depends on the secondary crosslinking temperature and the shape of the product. At 140° C., for example, the secondary crosslinking time is preferably 10 minutes to 15 hours, more preferably 10 minutes to 12 hours, further preferably 30 minutes to 8 hours, and still more preferably 30 minutes to 4 hours. The secondary crosslinking can be performed using an inert oven, a vacuum oven or the like in a batch method, or can be performed using a conveyor oven or the like in a continuous method.

Particularly in the case where the medical rubber according to the present invention is obtained by crosslinking an ethylene-propylene-diene rubber (EPDM) in the presence of a polyfunctional monomer (B) and zinc white (C) by an organic peroxide (A) having no aromatic ring structure, and further performing secondary crosslinking, the medical rubber can be produced, for example, by a production method including a step 1 of kneading the components mentioned above, a step 2 of crosslinking a non-crosslinked rubber composition obtained in the step 1, and a step 3 of further performing secondary crosslinking on a crosslinked rubber obtained in the step 2.

The kneading in the step 1 can be performed using a known kneading machine or mixer such as an internal mixer (e.g., an intermix, a Banbury mixer, and a kneader), and an open roll mill.

A known crosslinking method can be applied to the crosslinking in the step 2. For example, crosslinking molding can be performed at a temperature of 150 to 220° C. for approximately 0.5 to 60 minutes by, for example, compression molding or transfer molding, which include a press process or the like, or injection molding.

The secondary crosslinking in the step 3 involves a heat treatment of the crosslinked rubber obtained in the step 2, and can decrease low molecular weight compounds such as residues and decomposition products of the polymer in the crosslinked rubber to enhance cleanliness. The heat treatment for secondary crosslinking can be performed using a known heat treatment apparatus such as an oven, and more specifically using an inert oven, a vacuum oven or the like in a batch method, or a conveyor oven or the like in a continuous method.

The secondary crosslinking is preferably performed at a high temperature for a long period of time, but degradation of the crosslinked rubber may then be promoted. For this reason, the secondary crosslinking temperature is preferably 160° C. or less, more preferably 150° C. or less, and still more preferably 140° C. or less. Meanwhile, the lower limit is not particularly limited. The lower limit is preferably 100° C. or more, and more preferably 110° C. or more. The secondary crosslinking time may be appropriately set at, for example, 15 minutes to 24 hours, depending on the secondary crosslinking temperature and the shape of the product. At 140° C., for example, the secondary crosslinking time is preferably 1 hour or more, and more preferably 2 hours or more. The secondary crosslinking time is desirably short from the viewpoint of degradation of the crosslinked rubber and economy. The secondary crosslinking time is preferably 12 hours or less, more preferably 8 hours or less, and still more preferably 4 hours or less.

The medical rubber according to the present invention can be used for rubber stoppers for drugs, syringe gaskets, syringe caps, and rubber stoppers for blood collection tubes, for example.

The medical rubber according to the present invention is in conformity with the standards for extractable substances specified in the Japanese Pharmacopoeia, Sixteenth Edition, and therefore can be used suitably.

### EXAMPLES

The present invention will be more specifically described referring to Examples, but the present invention will not be limited only to these.

Hereinafter, chemicals used in Examples and Comparative Examples will be collectively described.

EPDM (1): Mitsui EPT4021 made by Mitsui Chemicals, Inc. (diene (ethylidene norbornene) content: 8.1% by mass, ethylene content: 51% by mass, ML₁₊₄ (125° C.): 13)

EPDM (2): Mitsui EPT9090M made by Mitsui Chemicals, Inc. (diene (ethylidene norbornene) content: 14.0% by mass, ethylene content: 41% by mass, ML₁₊₄ (125° C.): 58)

EPDM (3): ESPRENE 532 made by Sumitomo Chemical Co., Ltd. (diene (ethylidene norbornene) content: 3.5% by mass, ethylene content: 51% by mass, ML₁₊₄ (125° C.): 81)

EPDM (4): Mitsui EPT1070 made by Mitsui Chemicals, Inc. (diene (dicyclopentadiene) content: 4.0% by mass, ethylene content: 48% by mass, ML₁₊₄ (125° C.): 48)

EPDM (5): Mitsui EPT3070 made by Mitsui Chemicals, Inc. (diene (ethylidene norbornene) content: 4.7% by mass, ethylene content: 58% by mass, ML₁+4 (125° C.): 47)

Triallyl isocyanurate: made by Nippon Kasei Chemical Company Limited

Carbon black: DIABLACK N550 made by Mitsubishi Chemical Corporation (N₂SA: 42 m²/g)

Stearic acid: stearic acid “Tsubaki” made by NOF CORPORATION

Organic peroxide (1): Trigonox D-T50 made by Kayaku Akzo Corporation (2,2-di(tert-butylperoxy)butane)

Organic peroxide (2): PERHEXA V40 made by NOF CORPORATION (n-butyl-4,4-di(tert-butylperoxy)valerate) (purity: 40%)

Organic peroxide (3): PERBUTYL E made by NOF CORPORATION (t-butyl peroxy 2-ethylhexyl monocarbonate)

Organic peroxide (4): PERBUTYL L made by NOF CORPORATION (t-butyl peroxylaurate)

Organic peroxide (5): PERBUTYL D made by NOF CORPORATION (di-tert-butyl peroxide)

Organic peroxide (6): PERCUMYL D made by NOF CORPORATION (dicumyl peroxide; containing an aromatic ring structure)

Organic peroxide (7): PERBUTYL C made by NOF CORPORATION (tert-butyl cumyl peroxide; containing an aromatic ring structure)

Filler: MISTRON VAPOR made by Nihon Mistron Co., Ltd.

Oil: Diana Process Oil PW380 made by Idemitsu Kosan Co., Ltd.

Zinc oxide: zinc oxide #2 made by Mitsui Mining & Smelting Co., Ltd.

Zinc white: zinc white No. 2 made by Mitsui Mining & Smelting Co., Ltd. (particle size: 0.5 μm)

### Examples and Comparative Examples

### (Diaphragms and Gaskets)

### (Kneading)

The materials other than the organic peroxide were mixed using a pressurized kneader at a temperature of 80° C. and a rotation of 40 rpm for 10 minutes or more, and then discharged when the temperature reached 120° C. The obtained composition was kneaded together with the organic peroxide in an open roll mill at 60° C. for approximately 5 minutes, whereby a non-crosslinked rubber composition was obtained.

### (Molding)

The composition obtained by kneading was crosslinking molded at 150° C. for 30 minutes using a press to obtain a crosslinked rubber for testing.

### (Secondary Crosslinking)

The crosslinked rubber was placed in an inert oven and subjected to secondary crosslinking at 140° C. for 1 hour to obtain a secondarily crosslinked rubber for testing.

The rubbers obtained in the production method (crosslinked rubbers and secondarily crosslinked rubbers) were evaluated as follows. The results of diaphragms are shown in Table 1, and the results of gaskets are shown in Table 2.

### (Hardness)

According to JIS K6253-3, the type A durometer hardness was measured.

### (Compression Set)

According to JIS K6262:2006, the compression set was measured by the following method.

A cylindrical test piece having a diameter of 29 mm and a thickness of 12.5 mm was held with a jig, compressed 25%, and heat treated at 120° C. for 22 hours. The test piece was left to cool at room temperature for 2 hours while the test piece remained compressed. Then, the jig was removed. After 30 minutes, the thickness of the test piece was measured and the compression set was calculated. A smaller value thereof indicates a smaller residual strain and a better test result.

### (Durability Test)

A durability test was performed using a 2-Port N.C. Solenoid Valve KL204 made by Danaher Corporation. A diaphragm having the same shape as that of diaphragm products was prepared, and dry run 10,000,000 times at room temperature and 5 Hz to perform a durability test. After the durability test, air was flowed at 0.3 MPa, and the diaphragm was checked for leakage by measuring the pressure loss of the air after 5 minutes. The diaphragm was rated as bad (x) if the pressure reduction was more than 15%, good (◯) if the pressure reduction was 15% or less, and very good ( [symbol] ) if the pressure reduction was 10% or less.

### <Tests for Extractable Substances>

According to the Test for Rubber Closure for Aqueous Infusions in the Japanese Pharmacopoeia, measurement was performed as follows. The samples were rated as good (◯) if they met the test standard, and bad (x) if they did not meet the standard.

A test solution was prepared as follows. The slab sheet having a thickness of 2 mm was washed with water, dried at room temperature, and placed in a hard glass container. Thereto, water was added in an amount 10 times the weight of the sample, and a proper stopper was put on. The hard glass container was heated for 1 hour in an autoclave heated to 121° C., and then removed. The container was left until the temperature of the container reached room temperature. Then, the sheet was quickly removed. The obtained solution was used as a test solution. A blank test solution was separately prepared by the same method, except that only water without the pressed sheet was put into the container.

### (Transmittance)

The transmittances at a wavelength of 430 nm and at a wavelength of 650 nm were measured with a path length of 10 mm using the blank test solution as control. The test solution having a transmittance of 99.0% or more is in conformity with the standard.

### (Foaming)

A volume of 5 mL of the test solution was placed in a stoppered test tube having an inner diameter of approximately 15 mm and a length of approximately 200 mm, and vigorously shaken and mixed for 3 minutes. Then, if the foam formed almost completely disappeared within 3 minutes, the test solution is in conformity with the standard.

(pH)

A volume of 20 mL of the test solution and 20 mL of the blank test solution were prepared. To each solution was added 1.0 mL of a solution prepared by dissolving 1.0 g of potassium chloride in water to give 1000 mL, and the pH of the two solutions was measured. If the difference in pH between the two solutions is 1.0 or less, the test solution is in conformity with the standard.

### (Zinc)

3-Fold diluted nitric acid was added to 10.0 mL of the test solution to prepare 20 mL of a sample solution. 3-Fold diluted nitric acid was added to 1.0 mL of a standard zinc solution for atomic absorption spectrophotometry to prepare 20 mL of a standard solution. Testing was performed by atomic absorption spectrophotometry under the following conditions. If the absorbance of the sample solution is equal to or less than the absorbance of the standard solution, the test solution is in conformity with the standard.

Here, the standard zinc solution for atomic absorption spectrophotometry is a solution prepared by adding water to 10 mL of a standard zinc stock solution to make 1000 mL, and 1 mL of the standard zinc solution contains 0.01 mg of zinc.

Measurement conditions:

Gas used: acetylene;

Combustion-supporting gas: air;

Lamp: zinc hollow cathode lamp;

Wavelength: 213.9 nm.

### (Potassium Permanganate-Reducing Substances)

A volume of 100 mL of the test solution was placed in a stoppered conical flask, and 10.0 mL of a 0.002 mol/L potassium permanganate solution and 5 mL of dilute sulfuric acid were added. The resulting solution was boiled for 3 minutes, and cooled. Then, 0.10 g of potassium iodide was added to the solution, the flask was tightly sealed, and the solution was shaken and mixed, and then left as it was for 10 minutes. Then, the solution was titrated with 0.01 mol/L sodium thiosulfate (indicator: 5 drops of a starch test solution). Separately, 100 mL of the blank test solution was used and the same operation was performed. The difference in the consumption amount of the 0.002 mol/L potassium permanganate solution between the two solutions was measured. If the difference in the consumption amount of the 0.01 N potassium permanganate solution is 2.0 mL or less, the test solution is in conformity with the standard.

### (Residue on Evaporation)

A volume of 100 mL of the test solution was prepared, and evaporated to dryness on a water bath. The residue was dried at 105° C. for 1 hour, and the weight of the dried residue was measured. If the weight of the residue is 2.0 mg or less, the test solution is in conformity with the standard.

### (Ultraviolet Absorption)

A test was performed on the test solution against the blank test solution according to an absorbance measurement method. If the absorbance at a wavelength of 220 to 350 nm is 0.20 or less, the test solution is in conformity with the standard.

Among the tests for extractable substances after the secondary crosslinking, only the potassium permanganate-reducing substances and the ultraviolet absorption are shown in the table. The results of other test items not shown in the table are in conformity with the standards.

TABLE 1
(Diaphragms)

| | Example | Example | Example | Example | Example | Example | Example | Example | Example | Example |
|---|---|---|---|---|---|---|---|---|---|---|
| | 1 | 2 | 3 | 4 | 5 | 6 | 7 | 8 | 9 | 10 |
| EPDM (1) | | | | | | | 100 | 100 | 100 | 100 |
| EPDM (2) | | | 25 | 20 | 60 | 100 | | | | |
| EPDM (3) | | | | | 40 | | | | | |
| EPDM (4) | 100 | | 75 | | | | | | | |
| EPDM (5) | | 100 | | 80 | | | | | | |
| Carbon black | 40 | 40 | 40 | 40 | 40 | 40 | 40 | 40 | 40 | 40 |
| Stearic acid | 0.1 | 0.1 | 0.1 | 0.1 | 0.1 | 0.1 | 0.1 | 0.1 | 0.1 | 0.1 |
| Organic peroxide (1) | 2 | 2 | 2 | 2 | 2 | 2 | 2 | | | |
| Organic peroxide (2) | | | | | | | | 2 | | |
| Organic peroxide (3) | | | | | | | | | 2 | |
| Organic peroxide (4) | | | | | | | | | | 2 |
| Organic peroxide (5) | | | | | | | | | | |
| Organic peroxide (6) | | | | | | | | | | |
| Organic peroxide (7) | | | | | | | | | | |
| Total diene content | 4 | 4.7 | 6.5 | 6.6 | 9.8 | 14 | 8.1 | 8.1 | 8.1 | 8.1 |
| Hardness | 47 | 50 | 54 | 56 | 62 | 66 | 61 | 57 | 58 | 57 |
| Compression set | 44 | 39 | 37 | 34 | 30 | 26 | 33 | 32 | 32 | 33 |
| Durability test | X | ◯ | ◯ | ⊚ | ⊚ | ⊚ | ⊚ | ⊚ | ⊚ | ⊚ |

Tests for extractable substances

| | | | | | | | | | | |
|---|---|---|---|---|---|---|---|---|---|---|
| Transmittance | ◯ | ◯ | ◯ | ◯ | ◯ | ◯ | ◯ | ◯ | ◯ | ◯ |
| Foaming | ◯ | ◯ | ◯ | ◯ | ◯ | ◯ | ◯ | ◯ | ◯ | ◯ |
| pH | ◯ | ◯ | ◯ | ◯ | ◯ | ◯ | ◯ | ◯ | ◯ | ◯ |
| Zinc | ◯ | ◯ | ◯ | ◯ | ◯ | ◯ | ◯ | ◯ | ◯ | ◯ |
| Residue on evaporation | ◯ | ◯ | ◯ | ◯ | ◯ | ◯ | ◯ | ◯ | ◯ | ◯ |
| Ultraviolet absorption | ◯ | ◯ | ◯ | ◯ | ◯ | ◯ | ◯ | ◯ | ◯ | ◯ |

Tests for extractable substances after secondary crosslinking

| | | | | | | | | | | |
|---|---|---|---|---|---|---|---|---|---|---|
| Potassium | ◯ | ◯ | ◯ | ◯ | ◯ | ◯ | ◯ | ◯ | ◯ | ◯ |
| permanganate- | | | | | | | | | | |
| reducing substances | | | | | | | | | | |
| Ultraviolet absorption | ◯ | ◯ | ◯ | ◯ | ◯ | ◯ | ◯ | ◯ | ◯ | ◯ |

| | Example | Example | Example | Exampla | Example | Comparative | Comparative |
|---|---|---|---|---|---|---|---|
| | 11 | 12 | 13 | 14 | 15 | Example 1 | Example 2 |
| EPDM (1) | 100 | 100 | 100 | 100 | 100 | 100 | 100 |
| EPDM (2) | | | | | | | |
| EPDM (3) | | | | | | | |
| EPDM (4) | | | | | | | |
| EPDM (5) | | | | | | | |
| Carbon black | 40 | 40 | 40 | 15 | 70 | 40 | 40 |
| Stearic acid | 0.1 | 0.1 | 0.1 | 0.1 | 0.1 | 0.1 | 0.1 |
| Organic peroxide (1) | | 0.2 | 12 | 2 | 2 | | |
| Organic peroxide (2) | | | | | | | |
| Organic peroxide (3) | | | | | | | |
| Organic peroxide (4) | | | | | | | |
| Organic peroxide (5) | 2 | | | | | | |
| Organic peroxide (6) | | | | | | 2 | |
| Organic peroxide (7) | | | | | | | 2 |
| Total diene content | 8.1 | 8.1 | 8.1 | 8.1 | 8.1 | 8.1 | 8.1 |
| Hardness | 62 | 41 | 73 | 45 | 69 | 60 | 59 |
| Compression set | 31 | 41 | 15 | 35 | 29 | 30 | 31 |
| Durability test | ⊚ | ◯ | ◯ | ◯ | ◯ | ⊚ | ⊚ |

Tests for extractable substances

| | | | | | | | |
|---|---|---|---|---|---|---|---|
| Transmittance | ◯ | ◯ | ◯ | ◯ | ◯ | ◯ | ◯ |
| Foaming | ◯ | ◯ | ◯ | ◯ | ◯ | ◯ | ◯ |
| pH | ◯ | ◯ | ◯ | ◯ | ◯ | ◯ | ◯ |
| Zinc | ◯ | ◯ | ◯ | ◯ | ◯ | ◯ | ◯ |
| Residue on evaporation | ◯ | ◯ | ◯ | ◯ | ◯ | ◯ | ◯ |
| Ultraviolet absorption | ◯ | ◯ | ◯ | ◯ | ◯ | X | X |

Tests for extractable substances after secondary crosslinking

| | | | | | | | |
|---|---|---|---|---|---|---|---|
| Potassium | ◯ | ◯ | ◯ | ◯ | ◯ | X | X |
| permanganate- | | | | | | | |
| reducing substances | | | | | | | |
| Ultraviolet absorption | ◯ | ◯ | ◯ | ◯ | ◯ | X | X |

Formulation amount: part(s) by mass

In Comparative Examples 1 and 2 in which EPDM was crosslinked by an organic peroxide having an aromatic ring structure, the test solutions were not in conformity with the standards specified in the items of the potassium permanganate-reducing substances and the ultraviolet absorption in the tests for extractable substances. In contrast, in Examples in which EPDM was crosslinked by an organic peroxide (A) having no aromatic ring structure, the test solutions were in conformity with the standards specified in all the items in the tests for extractable substances. The rubbers in Examples also had excellent compression set resistance. Consequently, it is demonstrated that the rubber products for diaphragms containing no halogen atom in Examples are environmentally desirable, and also have excellent cleanliness and compression set resistance.

TABLE 2
(Gaskets)

| | Example | Example | Example | Example | Example | Comparative | Comparative |
|---|---|---|---|---|---|---|---|
| | 16 | 17 | 18 | 19 | 20 | Example 3 | Example 4 |
| EPDM (1) | 80 | 80 | 80 | 80 | 80 | 80 | 80 |
| EPDM (2) | 20 | 20 | 20 | 20 | 20 | 20 | 20 |
| Filler | 40 | 40 | 40 | 40 | 40 | 40 | 40 |
| Oil | 5 | 5 | 5 | 5 | 5 | 5 | 5 |
| Zinc oxide | 2 | 2 | 2 | 2 | 2 | 2 | 2 |
| Stearic acid | 0.1 | 0.1 | 0.1 | 0.1 | 0.1 | 0.1 | 0.1 |
| Organic peroxide (1) | 2 | 4 | | | | | |
| Organic peroxide (2) | | | 2 | | | | |
| Organic peroxide (3) | | | | 2 | | | |
| Organic peroxide (4) | | | | | 2 | | |
| Organic peroxide (6) | | | | | | 2 | |
| Organic peroxide (7) | | | | | | | 2 |
| Compression set | 29 | 27 | 31 | 30 | 31 | 30 | 30 |

Tests for extractable substances

| | | | | | | | |
|---|---|---|---|---|---|---|---|
| Transmittance | ◯ | ◯ | ◯ | ◯ | ◯ | ◯ | ◯ |
| Foaming | ◯ | ◯ | ◯ | ◯ | ◯ | ◯ | ◯ |
| pH | ◯ | ◯ | ◯ | ◯ | ◯ | ◯ | ◯ |
| Zinc | ◯ | ◯ | ◯ | ◯ | ◯ | ◯ | ◯ |
| Residue on evaporation | ◯ | ◯ | ◯ | ◯ | ◯ | ◯ | ◯ |
| Ultraviolet absorption | ◯ | ◯ | ◯ | ◯ | ◯ | X | X |

Tests for extractable substances after secondary crosslinking

| | | | | | | | |
|---|---|---|---|---|---|---|---|
| Potassium | ◯ | ◯ | ◯ | ◯ | ◯ | X | X |
| permanganate- | | | | | | | |
| reducing substances | | | | | | | |
| Ultraviolet absorption | ◯ | ◯ | ◯ | ◯ | ◯ | X | X |

Formulation amount: part(s) by mass

The gaskets, which included EPDM crosslinked by an organic peroxide (A) having no aromatic ring structure, also had the same effect as in the diaphragms.

### (Kneading)

The materials other than the organic peroxide were mixed using a pressurized kneader at a temperature of 80° C. and a rotation of 40 rpm for 10 minutes or more, and discharged when the temperature reached 120° C. The obtained composition was kneaded together with the organic peroxide using an open roll mill at 60° C. for approximately 5 minutes, whereby a non-crosslinked rubber composition was obtained.

### (Molding)

The composition obtained by kneading was crosslinking molded at 150° C. for 30 minutes using a press to obtain a crosslinked rubber.

### (Secondary Crosslinking)

The crosslinked rubber was placed in an inert oven and subjected to secondary crosslinking at 140° C. for 0.5 to 13 hours to obtain a secondarily crosslinked rubber for testing.

The thus obtained secondarily crosslinked rubbers were evaluated as follows. The results are shown in Table 3.

### (Hardness)

According to JIS K6253-3, the type A durometer hardness was measured.

### (Compression Set)

According to JIS K6262:2006, the compression set was measured by the following method.

A cylindrical test piece having a diameter of 29 mm and a thickness of 12.5 mm was held with a jig, and compressed 25% at 23° C. for 24 hours. The jig was then removed. After 30 minutes, the thickness of the test piece was measured and the compression set was calculated. It can be determined that a smaller value thereof indicates a smaller residual strain and a better test result. Then, in Examples and Comparative Examples, the relative value of compression set was determined where the compression set of the crosslinked rubber of Comparative Example 5, which was not subjected to secondary crosslinking, was 100. The test piece was rated as good if the relative value was less than 105, and bad if the relative value was 105 or more.

### <Tests for Extractable Substances>

According to the Test for Rubber Closure for Aqueous Infusions in the Japanese Pharmacopoeia, measurement was performed as follows. The samples were rated as good (◯) if they met the test standard, and bad (x) if they did not meet the standard.

A test solution was prepared as follows. The slab sheet having a thickness of 2 mm was washed with water, dried at room temperature, and placed in a hard glass container. Thereto, water was added in an amount 10 times the mass of the sample, and a proper stopper was put on. The hard glass container was heated for 1 hour in an autoclave heated to 121° C., and then removed. The container was left until the temperature of the container reached room temperature. Then, the sheet was quickly removed. The obtained solution was used as a test solution. A blank test solution was separately prepared by the same method, except that only water without the pressed sheet was put into the container.

### (Transmittance)

The transmittances at a wavelength of 430 nm and at a wavelength of 650 nm were measured with a path length of 10 mm using the blank test solution as control. The test solution having a transmittance of 99.0% or more is in conformity with the standard.

### (Foaming)

A volume of 5 mL of the test solution was placed in a stoppered test tube having an inner diameter of approximately 15 mm and a length of approximately 200 mm, and vigorously shaken and mixed for 3 minutes. Then, if the foam formed almost completely disappeared within 3 minutes, the test solution is in conformity with the standard.

(pH)

A volume of 20 mL of the test solution and 20 mL of the blank test solution were prepared. To each solution was added 1.0 mL of a solution prepared by dissolving 1.0 g of potassium chloride in water to give 1000 mL, and the pH of the two solutions was measured. If the difference in pH between the two solutions is 1.0 or less, the test solution is in conformity with the standard.

### (Zinc)

3-Fold diluted nitric acid was added to 10.0 mL of the test solution to prepare 20 mL of a sample solution. 3-Fold diluted nitric acid was added to 1.0 mL of a standard zinc solution for atomic absorption spectrophotometry to prepare 20 mL of a standard solution. Testing was performed by atomic absorption spectrophotometry under the following conditions. If the absorbance of the sample solution is equal to or less than the absorbance of the standard solution, the test solution is in conformity with the standard.

Here, the standard zinc solution for atomic absorption spectrophotometry is a solution prepared by adding water to 10 mL of a standard zinc stock solution to make 1000 mL, and 1 mL of the standard zinc solution contains 0.01 mg of zinc.

Measurement conditions:

Gas used: acetylene;

Combustion-supporting gas: air;

Lamp: zinc hollow cathode lamp;

Wavelength: 213.9 nm.

### (Potassium Permanganate-Reducing Substances)

A volume of 100 mL of the test solution was placed in a stoppered conical flask, and 10.0 mL of a 0.002 mol/L potassium permanganate solution and 5 mL of dilute sulfuric acid were added. The resulting solution was boiled for 3 minutes, and cooled. Then, 0.10 g of potassium iodide was added to the solution, the flask was tightly sealed, and the solution was shaken and mixed, and then left as it was for 10 minutes. Then, the solution was titrated with 0.01 mol/L sodium thiosulfate (indicator: 5 drops of a starch test solution). Separately, 100 mL of the blank test solution was used and the same operation was performed. The difference in the consumption amount of the 0.002 mol/L potassium permanganate solution between the two solutions was measured. If the difference in the consumption amount of the 0.01 N potassium permanganate solution is 2.0 mL or less, the test solution is in conformity with the standard.

### (Residue on Evaporation)

A volume of 100 mL of the test solution was prepared, and evaporated to dryness on a water bath. The residue was dried at 105° C. for 1 hour, and the mass of the dried residue was measured. If the mass of the residue is 2.0 mg or less, the test solution is in conformity with the standard.

### (Ultraviolet Absorption)

A test was performed on the test solution against the blank test solution according to an absorbance measurement method. If the absorbance at a wavelength of 220 to 350 nm is 0.20 or less, the test solution is in conformity with the standard.

TABLE 3

| | | Example | Example | Example | Example | Example | Example | Example | Example | Example | Example |
|---|---|---|---|---|---|---|---|---|---|---|---|
| | | 21 | 22 | 23 | 24 | 25 | 26 | 27 | 28 | 29 | 30 |
| EPDM (1) | | 100 | 100 | 100 | 100 | 100 | 100 | 100 | 100 | 100 | 100 |
| Triallyl isocyanurate | | 2 | 2 | 2 | 2 | 2 | 2 | 2 | 1 | 8 | 2 |
| Zinc white | | 4 | 4 | 4 | 4 | 4 | 4 | 4 | 4 | 4 | 1 |
| Carbon black | | 40 | 40 | 40 | 40 | 40 | 40 | 40 | 40 | 40 | 40 |
| Stearic acid | | 0.1 | 0.1 | 0.1 | 0.1 | 0.1 | 0.1 | 0.1 | 0.1 | 0.1 | 0.1 |
| Organic peroxide (1) | | 2 | | | | | 1 | 8 | 2 | 2 | 2 |
| Organic peroxide (2) | | | 2 | | | | | | | | |
| Organic peroxide (3) | | | | 2 | | | | | | | |
| Organic peroxide (4) | | | | | 2 | | | | | | |
| Organic peroxide (5) | | | | | | 2 | | | | | |
| Organic peroxide (6) | | | | | | | | | | | |
| Organic peroxide (7) | | | | | | | | | | | |
| Secondary crosslinking time | | 4 | 4 | 4 | 4 | 4 | 4 | 4 | 4 | 4 | 4 |
| Hardness | | 64 | 60 | 61 | 61 | 63 | 62 | 69 | 61 | 64 | 61 |
| Compression set | | 100 | 103 | 102 | 101 | 99 | 103 | 99 | 104 | 98 | 103 |
| Tests | Transmittance | ◯ | ◯ | ◯ | ◯ | ◯ | ◯ | ◯ | ◯ | ◯ | ◯ |
| for | Foaming | ◯ | ◯ | ◯ | ◯ | ◯ | ◯ | ◯ | ◯ | ◯ | ◯ |
| extract- | pH | ◯ | ◯ | ◯ | ◯ | ◯ | ◯ | ◯ | ◯ | ◯ | ◯ |
| able | Zinc | ◯ | ◯ | ◯ | ◯ | ◯ | ◯ | ◯ | ◯ | ◯ | ◯ |
| sub- | Potassium | ◯ | ◯ | ◯ | ◯ | ◯ | ◯ | ◯ | ◯ | ◯ | ◯ |
| stances | permanganate- | | | | | | | | | | |
| | reducing substances | | | | | | | | | | |
| | Residue on | ◯ | ◯ | ◯ | ◯ | ◯ | ◯ | ◯ | ◯ | ◯ | ◯ |
| | evaporation | | | | | | | | | | |
| | Ultraviolet | ◯ | ◯ | ◯ | ◯ | ◯ | ◯ | ◯ | ◯ | ◯ | ◯ |
| | absorption | | | | | | | | | | |

| | | Example | Example | Example | Example | Example | Comparative | Comparative | Comparative |
|---|---|---|---|---|---|---|---|---|---|
| | | 31 | 32 | 33 | 34 | 35 | Example 5 | Example 6 | Example 7 |
| EPDM (1) | | 100 | 100 | 100 | 100 | 100 | 100 | 100 | 100 |
| Triallyl isocyanurate | | 2 | 2 | 2 | | 2 | 2 | 2 | 2 |
| Zinc white | | 8 | 4 | 4 | 4 | | 4 | 4 | 4 |
| Carbon black | | 40 | 40 | 40 | 40 | 40 | 40 | 40 | 40 |
| Stearic acid | | 0.1 | 0.1 | 0.1 | 0.1 | 0.1 | 0.1 | 0.1 | 0.1 |
| Organic peroxide (1) | | 2 | 2 | 2 | 2 | 2 | | | |
| Organic peroxide (2) | | | | | | | | | |
| Organic peroxide (3) | | | | | | | | | |
| Organic peroxide (4) | | | | | | | | | |
| Organic peroxide (5) | | | | | | | | | |
| Organic peroxide (6) | | | | | | | 2 | 2 | |
| Organic peroxide (7) | | | | | | | | | 2 |
| Secondary crosslinking time | | 4 | 2 | 13 | 4 | 4 | 0 | 4 | 4 |
| Hardness | | 66 | 63 | 64 | 63 | 63 | 63 | 64 | 63 |
| Compression set | | 100 | 101 | 104 | 110 | 107 | 100 | 100 | 101 |
| Tests | Transmittance | ◯ | ◯ | ◯ | ◯ | ◯ | ◯ | ◯ | ◯ |
| for | Foaming | ◯ | ◯ | ◯ | ◯ | ◯ | ◯ | ◯ | ◯ |
| extract- | pH | ◯ | ◯ | ◯ | ◯ | ◯ | ◯ | ◯ | ◯ |
| able | Zinc | ◯ | ◯ | ◯ | ◯ | ◯ | ◯ | ◯ | ◯ |
| sub- | Potassium | ◯ | ◯ | ◯ | ◯ | ◯ | X | X | X |
| stances | permanganate- | | | | | | | | |
| | reducing substances | | | | | | | | |
| | Residue on | ◯ | ◯ | ◯ | ◯ | ◯ | ◯ | ◯ | ◯ |
| | evaporation | | | | | | | | |
| | Ultraviolet | ◯ | ◯ | ◯ | ◯ | ◯ | X | X | X |
| | absorption | | | | | | | | |

Formulation amount: part(s) by mass

In Examples in which EPDM, in the presence of a polyfunctional monomer and zinc white, was crosslinked by an organic peroxide having no aromatic ring structure and was further subjected to secondary crosslinking, the obtained rubbers exhibited good results in the tests for extractable substances, and also had excellent compression set resistance. Consequently, it is demonstrated that the medical rubbers containing no halogen atom in Examples are environmentally desirable, and also have excellent cleanliness and compression set resistance.

1. A medical rubber, comprising an ethylene-propylene-diene rubber crosslinked by an organic peroxide (A) having no aromatic ring structure.

2. The medical rubber according to claim 1,
which is subjected to secondary crosslinking.

3. The medical rubber according to claim 2,
wherein the medical rubber is obtained by crosslinking an ethylene-propylene-diene rubber in the presence of a polyfunctional monomer (B) and zinc white (C) by the organic peroxide (A) having no aromatic ring structure, and further performing secondary crosslinking.

4. The medical rubber according to claim 1,
wherein a diene component in the ethylene-propylene-diene rubber is derived from ethylidene norbornene.

5. The medical rubber according to claim 4,
wherein an ethylidene norbornene content is 6 to 14% by mass.

6. The medical rubber according to claim 1,
wherein the organic peroxide (A) is at least one selected from the group consisting of compounds respectively represented by the following formulas (1), (2), and (3):
(H₃C₃)₃C—O—O—R¹¹—O—O—C(CH₃)₃ (1)
wherein R¹¹ represents a saturated divalent hydrocarbon group optionally containing a substituent; 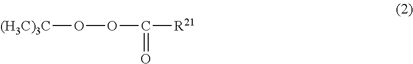
wherein R²¹ represents a saturated monovalent hydrocarbon group or a saturated alkoxy group; and
(H₃C₃)₃C—O—O—C(CH₃)₃ (3).

7. The medical rubber according to claim 6,
wherein the substituent is a group represented by —C(═O)—O—R¹² wherein R¹² is a saturated monovalent hydrocarbon group.

8. The medical rubber according to claim 1,
wherein 0.3 to 15 parts by mass of the organic peroxide (A) is contained per 100 parts by mass of the ethylene-propylene-diene rubber.

9. The medical rubber according to claim 3,
wherein the polyfunctional monomer (B) is at least one selected from the group consisting of di- or triallyl compounds, di(meth)acrylates, tri(meth)acrylates, divinyl compounds, and maleimide compounds.

10. The medical rubber according to claim 3,
wherein 0.5 to 10 parts by mass of the polyfunctional monomer (B) is contained per 100 parts by mass of the ethylene-propylene-diene rubber.

11. The medical rubber according to claim 3,
wherein 0.5 to 10 parts by mass of the zinc white (C) is contained per 100 parts by mass of the ethylene-propylene-diene rubber.

12. The medical rubber according to claim 2,
which is obtained by performing the secondary crosslinking for 1 hour or more.

13. The medical rubber according to claim 1,
which is in conformity with the standards for extractable substances specified in the Japanese Pharmacopoeia, Sixteenth Edition.
